(19) United States
(12) Patent Application Publication
(10) Pub. No.: US 2004/0236297 A1
(43) Pub. Date: Nov. 25, 2004

(54) Thin, flexible sanitary napkin having a compression resistant absorbent structure

(76) Inventors: Brian Michael Drzewiecki, Princeton, NJ (US); James P. Barr, East Amwell, NJ (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/444,443

(22) Filed: May 23, 2003

(51) Int. Cl. A61F 13/15; A61F 13/20

(52) U.S. Cl. 604/385.01; 604/367

(57) ABSTRACT

A thin, flexible sanitary napkin comprising an absorbent means having a compression resistance greater than about 110 kg/cm³, a flexural resistance of less than about 400 grams a test capacity of at least about 5.0 grams and a total capacity of at least about 10 grams.

### FIELD OF THE INVENTION

The present invention relates to sanitary napkins for feminine hygiene. Particularly, the present invention concerns thin, flexible sanitary napkins comprising a compression resistant absorbent structure and offering enhanced fit and comfort through a construction which promotes a continuously self-conforming anatomical cooperation of the sanitary napkin to the wearer to yield a highly effective absorbent device.

### BACKGROUND OF THE INVENTION

All manner and variety of sanitary napkins configured for the absorption of body fluids such as menses, urine and feces are, of course, well known. With respect to feminine protection devices, the art has offered two basic types; sanitary napkins have been developed for external wear about the pudendal region while tampons have been developed for internal wear within the vaginal cavity for interruption of menstrual flow therefrom.

Sanitary napkins find wide and varied use in absorbing and trapping body fluids and maintaining body surfaces in a state of dryness and comfort. These napkins generally include a liquid permeable layer which is placed nearest the body surface of the wearer and which may be an absorbent material serving as the main repository for liquid absorbed by the article. The napkin may be a multi-layer article having a liquid permeable layer, a separate absorbent material underlying the liquid permeable layer and a liquid impermeable layer impervious to liquid absorbed into the absorbent material and serving as a protective barrier between the absorbent material and the wearer's clothing. The napkins may also include an acquisition or transfer layer with a relatively open structure having a relatively high void volume for accepting and transporting the liquid to the absorbent material. The absorbent material typically has a high liquid absorption capacity relative to the liquid permeable layer and transfer layer and can be made from materials such as wood pulp, creped cellulose wadding, absorbent foams and sponges, polymeric fibers, aqueous liquid absorbing polymers and combinations of these materials.

At least two general classes of sanitary napkin pertinent to the present invention exist. One such class is for the absorption of medium to high menstrual flows. These sanitary napkins offer a fairly high absorptive capacity.

Such sanitary napkins theoretically have a high absorptive capacity, however, when the sanitary napkin is worn and subjected to the compressive forces of the wearer's thighs and pudendal region, the fluff pulp core simply compacts or compresses through the thickness and width of the core or bunches into an arbitrary, but generally rope-like shape. Such napkins commonly shift from their original placement so that after only a short wearing time, the napkin might only partially, if at all, be beneath the wearer's vaginal orifice or vestibule. Thus, in use, these sanitary napkins sometimes offer very little absorption. Further, the rolling and twisting of these napkins may create soiling on the wearer's panties and skin surfaces. In addition, the bulkiness of these napkins causes a high degree of wearing awareness and may make them quite obtrusive when worn with tight fitting slacks, body suits or bathing suits.

These conventional sanitary napkins have an absorbent material formed of cellulosic fibers such as cotton, wood pulp or rayon, mixtures of cellulosic and synthetic fibers such as polyethylene/polyester bicomponent fibers. These napkins, in order to provide the requisite absorbency, are generally fairly thick (>5 mm). The thickness of such an article can cause a high degree of wearing awareness, frequently resulting in user discomfort and/or poor fit between wearer's body surface and clothing. Although somewhat flexible and conforming to the body, thick sanitary napkins are compressible and frequently tend to deform or bunch in use, with further discomfort to the wearer and the possibility of loss of absorbency where it is most needed. When a napkin with this absorbent material is subjected to a compressive load typically experienced in use, the structure can collapse resulting in liquid being expressed from the absorbent material and rewetting the layers through which liquid was originally routed to the absorbent structure. Since typical transfer layers and liquid permeable body surface faceable layers are generally made from materials with little absorption capacity, the liquid expelled from the absorbent material will tend to reside next to the body surface of the wearer resulting in discomfort and possible staining of the wearer's garments.

One solution to this problem has been to incorporate aqueous liquid absorbing polymers, also known as superabsorbents, into the absorbent material. The development of materials having a high aqueous liquid absorption capacity per unit volume has allowed the previously required overall thickness of sanitary napkins to be substantially reduced providing products which are less obtrusive to wear. Combination of aqueous liquid absorbing polymers with fibrous absorbents has resulted in development of sanitary napkins which are relatively thin, i.e. less than 3 mm, and which generally retain absorbed fluid when subjected to compressive loads encountered in wearing the article. For example, U.S. Pat. No. 4,950,264 to Osborn, III discloses a relatively thin, i.e. caliper less than 2.6 mm, sanitary napkin having a generally compressible absorbent means comprising conventional absorbent materials such as pulp fluff and superabsorbent particles, the sanitary napkin having a flexural resistance of less than about 300 grams, a test capacity of at least about 8.0 grams and a total capacity of at least about 20.0 grams. Similarly, U.S. Pat. No. 4,217,901 to Bradstreet and Roller discloses a thin sanitary napkin having a caliper from about 3.0 to 7.0 mm and comprising a densified absorbent layer containing particulate superabsorbent and being resistant only to side compression, i.e., in the transverse direction of the sanitary napkin.

Compressible absorbent structures can collapse in use causing shifting of the absorbent means and/or bunching and twisting of the sanitary napkin in a user's undergarment or against a user's body. This can result in liquid not being absorbed and remaining on the body surface of the user and/or possibly staining the users' garments.

Conventional absorbent articles for feminine hygiene generally include a liquid permeable layer which is placed nearest the body surface of the wearer and which may be an absorbent material serving as the main repository for liquid absorbed by the article. The article may be a multi-layer article having a liquid permeable layer, a separate aqueous liquid absorbing material underlying the liquid permeable layer and a liquid impermeable layer impervious to liquid absorbed into the absorbent material and serving as a protective barrier between the absorbent material and the wearer's clothing. The articles may also include an acquisition or transfer layer with a relatively open structure having a relatively high void volume for accepting and transporting the liquid to the absorbent material. The aqueous liquid absorbing material typically has a high liquid absorption capacity relative to the liquid permeable layer and transfer layer and can be made from materials such as wood pulp, creped cellulose wadding, absorbent foams and sponges, polymeric fibers, aqueous liquid absorbing polymers and combinations of these materials.

A second class of conventional sanitary napkins are intended for light or low menstrual flows and are commonly referred to as pantiliners. Sanitary napkins designed for interlabial use or for use with specialty undergarments such as thong underwear may also be included in this class. Sanitary napkins of this class, as a group, are thinner, somewhat more flexible and generally more comfortable than those of the first class, however, they generally lack the absorptive capacity of the conventional napkins of the first class.

Therefore, there exists a real consumer need for a sanitary napkin which is thin, flexible and compression resistant, thereby offering enhanced fit and comfort, yet having a fluid capacity great enough for use with medium to high menstrual flows thereby allaying consumer fears of leakage and staining.

Therefore, it is an object of the present invention to provide a sanitary napkin which is thin, flexible and compression resistant in the Z direction and which is absorbent enough to absorb and contain medium to high menstrual flows. The resulting sanitary napkin is conformable and comfortable, freely adaptable both to the undergarment and to the body of the user.

These and other objects of the present invention will be more readily apparent when considered in reference to the following description and when taken in conjunction with the accompanying drawings.

### SUMMARY OF THE INVENTION

In accordance with a first embodiment of the present invention there has been provided a sanitary napkin for feminine hygiene adapted for placement in a crotch portion of an undergarment, having a body faceable surface and a garment faceable surface comprising a liquid permeable cover layer, a liquid impermeable barrier means and an absorbent means between the cover layer and the barrier means. The absorbent means has a compression resistance greater than about 110 kg/cm³; and the sanitary napkin has a flexural resistance of less than about 400.0 grams, a test capacity of at least about 5.0 grams, and a total capacity of at least about 10.0 grams. The sanitary napkin is relatively highly flexible and has a capacity great enough to handle medium to high menstrual flows.

In accordance with another embodiment of the present invention, there has been provided a sanitary napkin for feminine hygiene adapted for placement in a crotch portion of an undergarment and having a longitudinal axis. The sanitary napkin comprises a liquid permeable body faceable layer, a liquid impermeable garment faceable layer, an absorbent means positioned between said body faceable layer and said garment faceable layer and a peripheral zone aligned adjacent to one of said longitudinal edges of said absorbent means. The absorbent means extends longitudinally along the longitudinal axis of the sanitary napkin to form a central absorbent zone aligned along the longitudinal axis of said sanitary napkin and has two opposing longitudinal edges and two opposing transverse edges. The peripheral zone comprises the body faceable layer, the garment faceable layer and an absorbent material therebetween. The peripheral zone being characterized by an absorbency greater than about 5 g/g and a thickness less than about 4 mm and a compression resistance greater than about 110 kg/cm³. The central absorbent zone and the peripheral zone have a respective Gurley stiffness wherein the Gurley stiffness of the peripheral zone is less than the Gurley stiffness of the central absorbent zone.

Other aspects and features of the present invention will become apparent to those ordinarily skilled in the art upon review of the following description of specific embodiments of the invention in conjunction with the accompanying drawings.

### BRIEF DESCRIPTION OF THE DRAWINGS

While the specification concludes with claims particularly pointing out and distinctly claiming the present invention, it is believed that the present invention will be better understood from the following description in conjunction with the following drawings, in which like reference numbers identify identical elements and component thicknesses have been exaggerated for ease of illustration wherein:

FIG. 1 is a top plan view of an embodiment of the present invention with portions being torn away to show underlying structure.

FIG. 2 is an end view of the embodiment shown in FIG. 1 taken along line 2-2′ of FIG. 1.

FIG. 3 is a top plan view of another embodiment of the present invention with portions being torn away to show underlying structure.

FIG. 4 is an end view of the embodiment shown in FIG. 3 taken along line 3-3 ′ of FIG. 3.

### DETAILED DESCRIPTION OF THE INVENTION

The present invention relates to sanitary napkins for feminine hygiene and in particular to sanitary napkins which are thin, flexible and have a compression resistant absorbent means. Sanitary napkins of the invention comprise an absorbent means that is formed from a compression resistant absorbent material that offers enhanced fit and comfort through a construction which promotes a continuously self-conforming anatomical cooperation of the sanitary napkin to the wearer to yield a highly effective and unobtrusive absorbent device.

As used herein, the term “sanitary napkin” refers to an article which is worn by females adjacent to the pudendal region and which is intended to absorb and contain the various exudates which are discharged from the body (e.g., blood, menses and urine) and which is intended to be discarded after a single use (i.e., it is not intended to be laundered or otherwise restored or reused). As used herein, the term “pudendal” refers to the externally visible female genitalia and is limited to the labia majora, the labia minora, the clitoris, and the vestibule. Interlabial devices which reside partially within and partially external of the wearer's vestibule are also within the scope of this invention. Sanitary napkins designed to fit specialty undergarments such as thong underwear are also included.

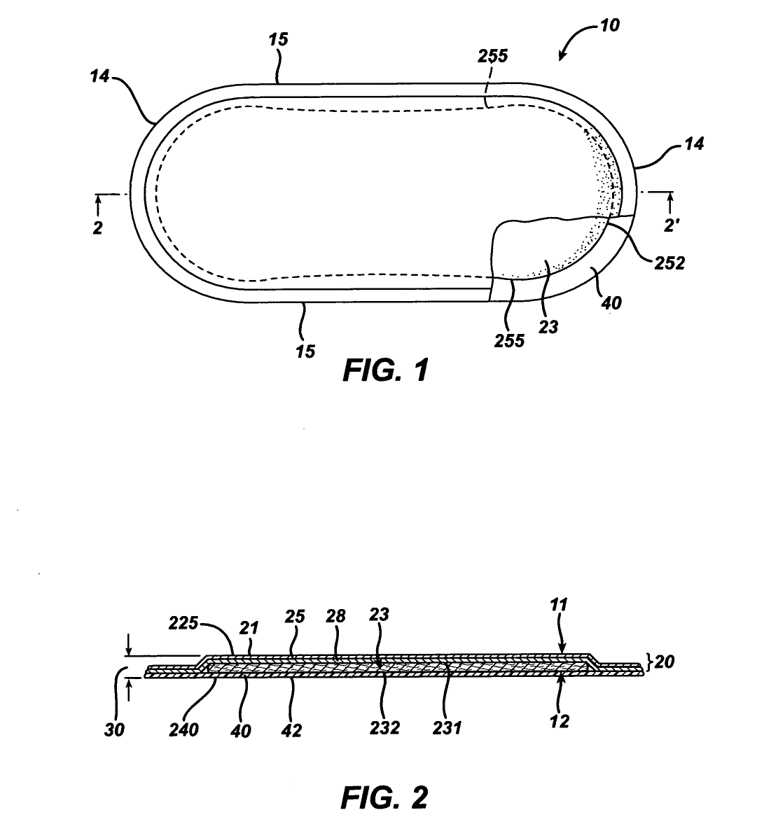

Referring to FIGS. 1 and 2, there is shown an embodiment of the present invention. In FIGS. 1 and 2, sanitary napkin 10 comprises an absorbent means 20 and a liquid impermeable barrier means 40. The absorbent means 20 may be any means which is generally conformable, non-irritating to the wearer's skin, capable of absorbing and containing body exudates such as menses, blood and urine and has a compression resistance greater than about 110 kg/cm³. The absorbent means 20 has a first major surface 11 and a second major surface 12, shown in FIG. 2. The barrier means 40 is adjacent the second major surface 12 of the absorbent means 20. The barrier means 40 may be any means which is flexible and liquid impervious and which prevents the exudates absorbed and contained in the absorbent means 20 from wetting articles which contact the sanitary napkin 10 such as an undergarment. The sanitary napkin 10 has a flexural resistance less than about 400 grams, a test capacity of at least about 5.0 grams and a total capacity of at least about 10 grams.

The sanitary napkin 10 of the present invention is highly flexible and conforms very well to the various shapes of the female urogenital region and thus has a low flexural resistance. The term “flexible” refers to the ability of the sanitary napkin to conform to the contours of the human body and/or the wearer's undergarments and may be defined as the flexural resistance or as the Gurley stiffness. The flexural resistance of a sanitary napkin is another measure of the stiffness of a sanitary napkin and is measured by peak bending stiffness. Sanitary napkins of the present invention that are highly flexible and conformable will have a flexural resistance of less than about 400 grams, preferably less than about 250 grams, more preferably less than about 175 grams and most preferably less than about 130 grams. Flexural resistance (often referred to as Peak bending stiffness) is determined by a test which is modeled after the ASTM D 4032-82 CIRCULAR BEND PROCEDURE, the procedure being considerably modified and performed as detailed in the Test Method section. The CIRCULAR BEND PROCEDURE is a simultaneous multi-directional deformation of a material in which one face of specimen becomes concave and the other face become convex. The CIRCULAR BEND PROCEDURE gives a force value related to flexural resistance, simultaneously averaging stiffness in all directions.

In general, the lower the Gurley stiffness value, the more flexible the material. The procedure for measuring Gurley stiffness is given in the Test Methods section. The sanitary napkins of the present invention have a Gurley Stiffness less than about 400 mg, preferably less than about 250 mg and even more preferably less than about 100 mg.

Extremely flexible structures can be referred to as drapeable. The terms “drapeable” and “drapability” are used interchangeably and mean having a flexural resistance of about 35 grams (g) or less as tested by the Modified Circular Bend Test, ASTM 4032-82 as set forth in the Example section of U.S. patent application Ser. No. 09/477,244 to Brisebois, et al, filed Jan. 4, 2000 and incorporated herein by reference. Drapeable articles comprising the aqueous liquid absorbing material of the present invention have also been found to have a flexural resistance of about 35 g or less.

Compression resistance is a measure of the compressibility of an absorbent material wherein the more resistant to a compressive force, the higher the compression resistance value. A compression resistant sanitary napkin is constructed of materials that do not collapse readily when subjected to an applied load, such as that incurred when a sanitary napkin is worn. As a result, the sanitary napkin will resist twisting, bunching and collapse in the Z direction. The sanitary napkin of the present invention has a compression resistance greater than about 110 kg/cm³ and preferably greater than 250 kg/cm³.

In the embodiment shown in FIGS. 1 and 2, the absorbent means 20 is comprised of a liquid permeable cover layer 25, an optional liquid permeable transfer layer 28, and an absorbent material 23.

The sanitary napkin 10 has a body faceable surface 225 which is generally defined by the liquid permeable cover layer 25 and a garment faceable surface 240, which is generally defined by the liquid impermeable barrier means 40, which define a caliper 30, shown in FIG. 2.

As used herein, the terminology “thin” refers to the thickness or caliper 30, of the sanitary napkin. Caliper is defined as the distance between the two opposing outermost surfaces, i.e., 225 and 240, of the sanitary napkin and is measured in an area of the article which contains the absorbent means 20. For example, in an embodiment in which an area of the article consists of cover, transfer layer, absorbent material and barrier means; while other areas of the article may consist solely of cover, transfer layer and barrier means; the caliper would be measured in the area containing the absorbent material. The procedure for measuring caliper is given in the Test Methods section. Because of the flexibility requirements of the sanitary napkin 10 of the present invention, it is likely that the sanitary napkin of the present invention will be relatively thin. It is preferred to keep the sanitary napkin 10 of the present invention thin so that said sanitary napkin will be unobtrusive and the user will have a low awareness of said sanitary napkin while it is being worn. Preferably the caliper of sanitary napkins of the present invention is less than about 4 mm. More preferably the caliper is less than about 3 mm and even more preferably the caliper is less than about 2 mm.

As previously mentioned, the sanitary napkin 10 of the present invention has a liquid capacity great enough to absorb medium to high menstrual flows. Two capacities, which, depending on the size of the sanitary napkin may be the same, are determinable: test capacity and total capacity. Preferably, the napkin 10 of the present invention has a test capacity of at least about 5.0 grams, more preferably of at least about 10.0 grams, and most preferably of at least about 15.0 grams. Preferably, the napkin 10 of the present invention has a total capacity of at least about 10.0 grams, more preferably of at least about 20.0 grams, and most preferably of at least about 30.0 grams. The procedure for determining Test Capacity and Total Capacity are given in the Test Methods Section.

Looking at some of the elements of the sanitary napkin 10 more specifically, the absorbent means 20 is formed from absorbent material 23 which may be any means which is generally conformable, non-irritating to the wearer's skin, capable of absorbing and containing body exudates and compression resistant. The absorbent means 20 has a first major surface 231, a second major surface 232, a pair of end edges 252 and a pair of side edges 255. The absorbent means 20 may be manufactured in a wide variety of sizes and shapes (e.g., rectangular, hourglass, etc.). A preferred shape of the absorbent means 20 is the substantially oval shape shown in FIG. 1. The absorbent means 20 is preferably symmetrically configured for ease of manufacture and so that no conscious effort is required by the wearer to properly place the sanitary napkin 10 in the direction it should be worn. The size of the absorbent means 20 may be varied to accommodate wearers ranging in size and also to provide a range in the expected amount of exudate fluid volume. The absorbent means 20 may be attached over the material's first or second major surfaces 231 and 232, respectively, to adjacent members such as the liquid permeable cover layer 25, optional liquid permeable transfer layer 28 or barrier means 40 by any of the means well known in the art, such as by spray-gluing or lines or spots of adhesive. Such attachment facilitates integrity of the absorbent materials in use so as to maintain an optimum degree of absorbency.

The absorbent means 20 may be manufactured from a wide variety of liquid absorbent materials 23 commonly used in disposable sanitary napkins, diapers, and other sanitary napkins provided of course that the result absorbent means 20 is generally conformable, non-irritating to the wearer's skin, capable of absorbing and containing body exudates and compression resistant. Non-limiting examples of suitable absorbent materials include comminuted wood pulp, creped cellulose wadding, absorbent foams, absorbent sponges, synthetic staple fibers, polymeric fibers, hydrogel-forming polymer gelling agents, or any equivalent materials or combinations of materials. Particularly preferred absorbent materials are polymeric gelling agents. Polymeric gelling agents are those materials which, upon contact with fluids (i.e., liquids) such as water or body fluids, imbibe such fluids and thereby form hydrogels. In this manner, fluid discharged into the absorbent material 23 can be acquired and held by the polymeric gelling agent, thereby providing the articles herein with enhanced absorbent capacity and/or improved fluid retention performance. The polymeric gelling agent which is employed in the absorbent material 23 will generally comprise particles of a substantially water-insoluble, slightly cross-linked, partially neutralized, hydrogel-forming polymer material.

The liquid impermeable barrier means 40 in embodiments of the present invention prevents and/or retards the through transfer of liquid but does not necessarily prevent the passage of gases. Suitable preferred materials include polyethylene or polypropylene films. Other materials that may be used as the liquid impermeable barrier means include films of polyesters, polyamides, ethylene vinyl acetate, polyvinyl chloride, polyvinylidene chloride, cellophane, nitrocellulose and cellulose acetate. Co-extruded and laminated combinations of the foregoing, wherein such combinations are permitted by the chemical and physical properties of the film, may be used. Liquid impermeable reticulated foams may also be used. Adhesive coatings, for positioning structure 10 in a user's undergarments, may also serve as the liquid impermeable layer.

Liquid impermeable layers that block or retard liquid permeation, but permit gases to transpire, i.e., “breathable barriers”, may also be used as the liquid impermeable barrier means 40. Single or multiple layers of microporous films, fabrics and combinations thereof, that provide a tortuous path, and/or whose surface characteristics provide a liquid repellent surface to the penetration of liquids may be used to provide such breathable barriers. A nonwoven web particularly useful as a breathable, liquid impermeable barrier means is a spunbond polypropylene web, providing a retarding effect, but not necessarily an absolute barrier, to liquid strikethrough.

The liquid permeable cover layer 25 is a body faceable layer, in use, which can pass liquid to the absorbent means 20. Liquid permeable cover layer 25 is preferably compliant, soft feeling, and non-irritating to a user's skin and preferably exhibits rapid fluid strikethrough and a reduced tendency to rewet, permitting bodily discharges to rapidly penetrate it and flow toward subsequent underlying absorbent means 20, while not allowing such discharges to flow back through the liquid permeable layer to the skin of the user. A suitable liquid permeable cover layer 25 may be manufactured from a wide range of materials including, but not limited to, woven and nonwoven fabrics, apertured formed polymeric films, porous foams, reticulated foams, reticulated thermoplastic films, and thermoplastic scrims. In addition, the liquid permeable cover layer may be constructed from a combination of one or more of the above materials, such as a composite layer of nonwoven and apertured, formed polymeric film. Particularly suitable liquid permeable materials for liquid impermeable cover layer 25 include a thermobonded multidenier (3 and 5 denier) polypropylene nonwoven web, and a through-air bonded nonwoven comprising polyethylene sheath and polypropylene core bi-component fibers. The liquid permeable cover layer 25 may employ adhesives on its body faceable surface for attaching the structure 10 directly to a user's skin. Examples of adhesives suitable for body attachment include, but are not limited to styrene-ethylene-butadiene-styrene oil gels, polyethylene glycol-poly-HEMA, and silicones such as Dow Corning #9600.

In a particularly preferred embodiment of the invention, shown in FIGS. 1 and 2, the absorbent material 23 comprises an aqueous liquid absorbing material. The aqueous liquid absorbing material (ALAM ) comprises an absorbent hot melt adhesive containing greater than about 1% by weight of an aqueous liquid absorbing polymer. Absorbent material 23 comprising ALAM has an absorbency greater than about 5 grams per gram as measured by the Tea Bag Method and a Gurley stiffness less than about 400 mg. In this preferred embodiment, the sanitary napkin 10 also has a Gurley stiffness less than 400 mg and has caliper 30 less than about 4 mm. Preferably, the sanitary napkin 10 has a caliper less than about 3 mm and more preferably less than about 2 mm.

In this preferred embodiment absorbent material 23 comprises a homogeneous mixture of a hot melt adhesive containing greater than about 1% by weight of an aqueous liquid absorbing polymer. The aqueous liquid absorbing material is capable of absorbing and trapping liquid within the aqueous liquid absorbing polymer but is not soluble in the liquids absorbed. The aqueous liquid absorbing material may be formed by blending about 10% to 50% of a block copolymer; about 20% to 80% of a tackifying resin and greater than about 1% of an aqueous liquid absorbing polymer in suitable adhesive processing equipment such as a melt mixer or extruder at a temperature above their respective melting points until uniformly mixed. The amount of aqueous liquid absorbing polymer will define the absorbency of the material; the more aqueous liquid absorbing polymer added, the greater the absorbency.

Suitable block copolymers for use in the invention include linear or radial co-polymer structures having the formula (A−B)ₓ, wherein block A is a polyvinylarene block, block B is a poly(monoalkenyl) block, x denotes the number of polymeric arms, and wherein x is an integer greater than or equal to one. Suitable block A polyvinylarenes include, but are not limited to Polystyrene, Polyalpha-methylstyrene, Polyvinyltoluene, and combinations thereof. Suitable Block B poly(monoalkenyl) blocks include, but are not limited to conjugated diene elastomers such as for example polybutadiene or polyisoprene or hydrogenated elastomers such as ethylene butylene or ethylene propylene or polyisobutylene, or combinations thereof. Commercial examples of these types of block copolymers include Kraton™ elastomers from Shell Chemical Company, Vector™ elastomers from Dexco, Solprene™ from Enichem Elastomers and Stereon™ from Firestone Tire & Rubber Co.

Suitable tackifying resins include natural and modified resins; glycerol and pentaerythritol esters of natural and modified resins; polyterpene resins; copolymers and terpolymers of natural terpenes; phenolic modified terpene resins and the hydrogenated derivatives thereof, aliphatic petroleum resins and the hydrogenated derivatives thereof; aromatic petroleum resin and the hydrogenated derivatives thereof; and aliphatic or aromatic petroleum resins and the hydrogenated derivatives thereof, and combinations thereof. Commercial examples of these types of resins include Foral® hydrogenated rosin ester, Staybelite® hydrogenated modified rosin, Poly-pale® polymerized rosin, Permalyn® rosin ester, Pentalyn® rosin ester, Adtac® oil extended hydrocarbon resin, Piccopale® aromatic hydrocarbon, Piccotac®, Hercotac® aromatic modified aliphatic hydrocarbon, Regalrez® cycloaliphatic resins, or Piccolyte® from Hercules, Eselementz® from Exxon Chemical aliphatic hydrocarbon and cycloaliphatic resins, Wingtack® from Goodyear Tire & Rubber Co. synthetic polyterpene resins including aromatic modified versions, Arkon® partially and fully hydrogenated aromatic resins from Arakawa Chemicals, Zonatac® styrenated terpene resin, Zonarez® rosin ester and Zonester® rosin ester from Arizona Chemical and Nevtac® aromatic modified aliphatic hydrocarbon from Neville Chemical Company.

Aqueous liquid absorbing polymers suitable for inclusion in the aqueous liquid absorbing material include thermoplastic hydrogels such as superabsorbent materials or thermoplastic polymeric compositions, which are formed from a water-soluble soft segment and one or more hard segments. The hard segment must be melt processable, i.e. at use temperature the hard segments in the polymer are below their melt temperature, and at process temperature, the hard segments are above their melting point temperature and below the decomposition temperature of either the other components of the hot-melt adhesive composition. The hard segment is substantially insoluble in water, and phase separates from the soft segment. Examples of suitable hard segments include, but are not limited to polyurethane, polyamides, polyesters, polyureas, and combinations thereof. Examples of suitable soft segments include, but are not limited to polyethylene oxide, polyvinyl alcohol, polyvinyl pyrrolidone, polyacrylamide, polysaccharide, polymaleic anhydride, random copolymers of polyethylene oxide and polypropylene-oxide and combinations thereof. The soft and hard segments may be covalently bonded together by means of urethane, amide, ester, or secondary urea linkages or combinations thereof. Examples of aqueous liquid absorbing thermoplastic polymeric compositions which are commercially available include hydrophilic polyurethane from Tyndale Plains-Hunter Ltd. and Aquacaulk® thermoplastic polymers from Sumitomo Seika Chemicals Co., Ltd. Suitable superabsorbent materials include any of the conventional superabsorbent particles or superabsorbent fibers which are commercially available today Examples are Aquakeep SA-70 and J-550P from Sumitomo Seika Chemicals Co., Ltd. The superabsorbent material is preferably a superabsorbent particle having an average particle size less than about 150 microns. More preferably the superabsorbent particles have an average particle size less than about 125 microns. Most preferably, the superabsorbent particles have an average particle size less than about 75 microns. The small particle size of the aqueous liquid absorbing polymer results in increased absorbent polymer exposure at the surface of the aqueous liquid absorbing material and is essential for attaining a homogeneous mixture and for facilitating processing the material through conventional hot melt adhesive application equipment.

Suitable plasticizers for use in the present invention generally will include any conventional plasticizers which decrease hardness and modulus, enhance pressure sensitive tack and reduce melt and solution viscosity. It is preferred that the plasticizer be water soluble or water dispersible or alternatively be a wax-like substance such as polyethylene glycol, glycerin, glycerol, polypropylene glycol, butylene glycol or sorbitol. An example of a preferred plastizer is Carbowax® polyethylene glycol from Union Carbide.

Suitable anti-oxidants for use in the present invention include any conventional anti-oxidants, and are preferably hindered phenols such as for example Ethanox 330w 1,3,5-trimethyl-2,4,6-tris(3,5-di-tert-butyl-4-hydroxybenzyl) benzene which is commercially available from the Ethyl Corporation.

In a particularly preferred embodiment, the aqueous liquid absorbing material comprises (by weight): about 10-50% block copolymer, preferably with a melt index greater than about 10, about 20-80% tackifying resin, preferably with a softening point less than about 105° C., greater than about 1% aqueous liquid absorbing polymer, about 0-40% plasitcizer with viscosity from 1-500 centipoise at application temperature, and about 0-2.0% antioxidant.

The aqueous liquid absorbing material may be applied to liquid impermeable barrier means 40 or to another layer of sanitary napkin 10, i.e. to optional liquid permeable transfer layer 28, to form absorbent means 20 using suitable conventional adhesive processing equipment such as a hot melt adhesive slot coating head, a hot melt adhesive swirl spray applicator (a commercial example of which is a Nordson Control Fiberization®), using a hot melt adhesive micro fiber applicator (commercial examples of these applicators include Nordson Control Coat®, ITW Dynafiber®, J&M Meltblown, and May Coating's Accufiber®), using a hot melt adhesive rotary screen applicator to create a pattern coating (examples of this equipment include Nordson and Kraemer rotary screen technology).

The total absorbent capacity of the absorbent material 23 should be compatible with the design exudate loading for the intended use of the sanitary napkin 10. That is, the total absorbent capacity of the absorbent material 23 may be varied to accommodate wearers ranging in the expected amount of exudate fluid volume. For instance, a different absorbent capacity may be utilized for sanitary napkins intended for daytime use as compared with those intended for nightime use, or for sanitary napkins intended for use by teenage females as compared with those intended for use by more mature women.

It should be noted that the scope of the present invention is not intended to extend to sanitary napkins which are void of any superabsorbent material.

Preferably, the liquid permeable cover layer 25 and the liquid impermeable barrier means 40 have length and width dimensions generally larger than the absorbent material 23 so that they extend beyond the edges 252 and 255 of the absorbent material 23 where they are associated together in a suitable manner. As used herein, the term “associated” encompasses configurations whereby a first member is directly joined to a second member and configurations whereby a first member is indirectly joined to a second member by affixing the first member to intermediate members which in turn are affixed to the second member. The extension of the liquid permeable cover layer 25 and/or the barrier means 40 beyond the absorbent material end edges 252 and the absorbent material side edges 255 of the absorbent material 23 form the sanitary napkin end edges 14 and the side edges 15, respectively, of the sanitary napkin 10. In a preferred embodiment, the barrier means 40 and the liquid permeable cover layer cover layer 25 have an oval shape and extend beyond the absorbent material 23 a distance of at least about 1.0 centimeter where they are joined directly to each other by attachment means as are well known in the art.

The attachment means may be, for example, a uniform continuous layer of adhesive a patterned layer of adhesive, or an array of separate lines or spots of adhesives.

For the sanitary napkin of the present invention, attachment means (not shown) for attaching the sanitary napkin to a wearer's undergarment may occupy portions of the garment faceable surface 42 of the liquid impermeable barrier means 40 and/or portions of the body faceable surface 21 of liquid permeable cover layer 25. Adhesives (not shown) may be applied to body faceable surface 21 for attaching the liquid permeable layer directly to a user's body. Examples of adhesives suitable for body attachment include, but are not limited to styrene-ethylene-butadiene-styrene oil gels, polyethylene glycol-poly-HEMA, and silicones such as Dow Corning #9600.

Alternatively, positioning adhesives, mechanical fasteners, or high coefficient of friction materials may be applied to surface 42 for releaseably adhering the sanitary napkin of FIGS. 1 and 2 to a user's garment. Or, the barrier layer itself may be constructed from a high coefficient of friction material, such as natural or synthetic rubber, thereby eliminating the need for additional material to provide undergarment attachment. Preferably, positioning adhesives are used to adhere the sanitary napkin 10 to a crotch portion of a user's undergarments. Positioning adhesives suitable for sanitary napkins of the present invention are well known in the art, one known class being styrenic block copolymers. Techniques used for applying the adhesives to the sanitary napkin include, but are not limited to slot coating, spraying, knife coating, extrusion coating, and transfer coating. The adhesives may also be foamed prior to application, such as by using commercially available equipment from the Nordson Corporation. Adhesives may be coated in continuous or in discrete patterns from emulsion or solution directly onto the product substrate or onto a release substrate to be subsequently transferred onto the article.

The sanitary napkin 10 of the present invention has a low flexural resistance. Thus, the sanitary napkin 10 of the present invention is highly flexible and conforms very well to the various shapes of the female urogenital region. Preferably, the sanitary napkin 10 of the present invention has a flexural resistance of less than about 400 grams still, more preferably less than about 250 grams, more preferably less than about 175 grams and most preferably less than about 130 grams.

The flexural resistance of a sanitary napkin is measured by peak bending stiffness. Peak bending stiffness is determined by a test which is modeled after the ASTM D 4032-82 CIRCULAR BEND PROCEDURE, the procedure being considerably modified and performed as detailed in the Test Method section. The CIRCULAR BEND PROCEDURE is a simultaneous multi-directional deformation of a material in which one face of a specimen becomes concave and the other face becomes convex. The CIRCULAR BEND PROCEDURE gives a force value related to flexural resistance, simultaneously averaging stiffness in all directions.

As previously mentioned, the sanitary napkin 10 of the present invention has a liquid capacity great enough to absorb medium to high menstrual flows. Two capacities, which, depending on the size of the sanitary napkin may be the same, are determinable: test capacity and total capacity. Preferably, the napkin 10 of the present invention has a test capacity of at least about 5.0 grams, more preferably of at least about 10.0 grams, and most preferably of at least about 18.0 grams. Preferably, the napkin 10 of the present invention has a total capacity of at least about 10.0 grams, more preferably of at least about 25.0 grams, and most preferably of at least about 40.0 grams. The method for determining Test and Total Capacity is given in the Test Methods Section.

Table 1 compares Gurley stiffness and Compression Resistance, as evaluated according to the methods given in the TEST METHODS section below, of an embodiment of a preferred sanitary napkin of the present invention comprising aqueous liquid absorbing material (ALAM) with exemplary prior art sanitary napkins. The Example 1 ALAM containing sanitary napkin consists of four layers: 30 gsm, 3 and 5 denier multidenier polypropylene nonwoven; 65 gsm ALAM and 0.9 mil pink polyethylene barrier film.

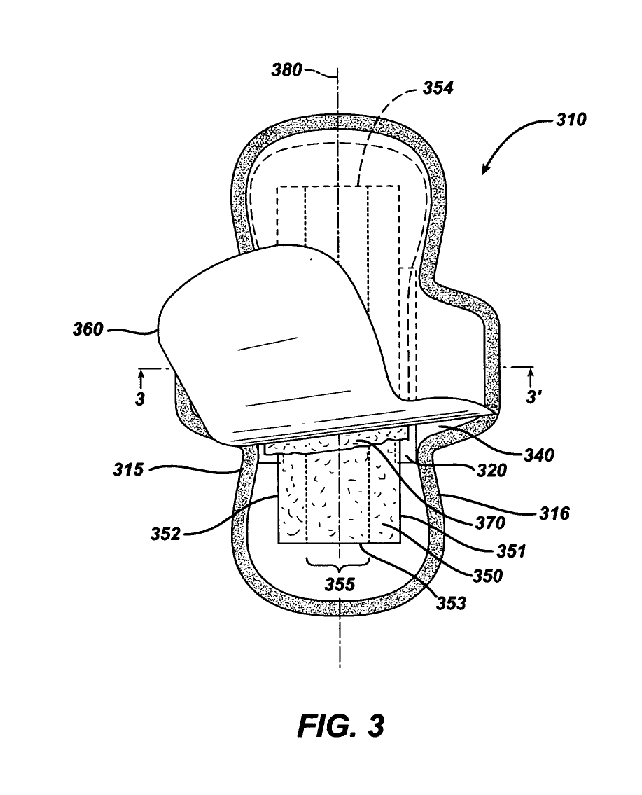

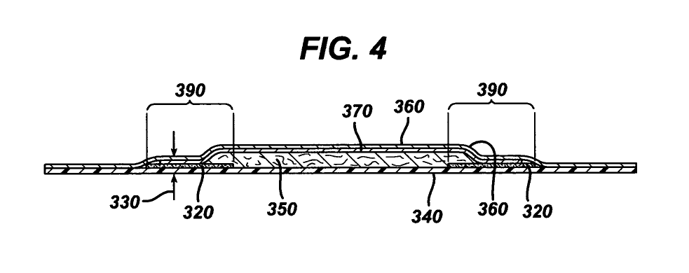

Referring to FIGS. 3 and 4, there is shown another embodiment of the present invention, a sanitary napkin 310 for feminine hygiene adapted for placement in a crotch portion of an undergarment and having a longitudinal axis 380. The sanitary napkin 310 comprising a liquid permeable body faceable layer 360, a liquid impermeable garment faceable layer 340, an optional transfer layer 370 disposed adjacent said body faceable layer, an absorbent means 350 positioned between said

TABLE 1

| | Gurley Stiffness, mg. | Compression, |
|---|---|---|
| Sample Description | avg. | kg/cm³ avg |
| EXAMPLE 1, | 18.02 | 259.80 |
| ALAM | | |
| STAYFREE ™ Ultra | 421.42 | 109.21 |
| Thin MAXI | | |
| Overnight with | | |
| WINGS | | |
| ALWAYS THIN | 233.08 | 77.01 |
| ULTRA ™ | | |
| Overnight, FLEXI- | | |
| WINGS ™ | | |
| Kotex ™ Ultra Thin | 755.24 | 57.67 |
| Overnight with | | |
| Wings | | |

transfer layer and said garment faceable layer and at least one peripheral zone 390 shown in FIG. 3. The absorbent means 350 has a longitudinally-extending central absorbent zone 355 aligned along the longitudinal axis 380 of said sanitary napkin 310 and has two opposing longitudinal edges 351, 352 parallel to and spaced apart from said longitudinal axis and two transverse edges 353, 354. The peripheral zone 390 is aligned adjacent to one of said longitudinal edges 351,352 of absorbent means 350 and is spaced apart from and is inward from the side edges 315,316 of the sanitary napkin 310. The peripheral zone 390 includes said body faceable layer 360, said optional transfer layer 370, said garment faceable layer 340 and absorbent material 320. Said peripheral zone 390 is characterized by an absorbency greater than about 5 g/g, a caliper 330 less than about 4 mm and a compression resistance greater than about 110 kg.cm³. The central absorbent zone 355 has a Gurley stiffness greater than the Gurley stiffness of the peripheral zone 390. The differences between Gurley stiffness of the central absorbent zone 355 and the peripheral absorbent zone 390 result in a sanitary napkin that is flexible and conforms readily to the crotch portion of an undergarment while resisting bunching and twisting.

Sanitary napkins according to the present invention provide at least two zones having different Gurley stiffness and which may have the same or different absorbency. Additionally, the peripheral zone has a compression resistance greater than about 110 kg.cm³ and more preferably greater than about 250 kg.cm³. This configuration, with differences in Gurley stiffness between the absorbent zone 355 and the peripheral zone 390, provides a sanitary napkin characterized by flexibility and resistance to compression in the Z direction as well as comfort and conformability for the user. The flexible and compression resistant peripheral zone is conformable and comfortable in use and is absorbent, without compressing or bunching which may compromise protection from leakage.

In sanitary napkins of the present invention, peripheral zone 390 preferably has a Gurley stiffness less than 200 mg, more preferably less than 100 mg and most preferable less than 50 mg. Preferably the difference in Gurley stiffness of the central absorbent zone and the peripheral zone is less than 500 mg, more preferably the difference is less than 200 mg, and most preferably the difference in Gurley stiffness of the central absorbent zone and the peripheral zone is at less than 50 mg. In a most preferred embodiment, the absorbent material 320 is aligned parallel and adjacent to said absorbent means 350 and is partially covered by the absorbent means 350 as shown in FIG. 4. This embodiment provides an extremely flexible, yet highly absorbent peripheral zone for absorbing fluid wicking along the sides of the sanitary napkin, thus reducing side leakage incidents in sanitary napkins with this construction while remaining very conformable and comfortable in use. In accordance with this embodiment, the absorbent means may be formed from conventional materials used to form absorbent materials in sanitary napkins. Attachment means (not shown) are discussed above in the description of FIGS. 1 and 2.

A representative, non-limiting list, of absorbents useful in the absorbent means 350 includes fibrous materials such as natural cellulosics, i.e. cotton and wood pulp; regenerated cellulosics, i.e. rayon and cellulose acetate; peat moss; hydrogel-forming polymers in the form of fibers or particles, commonly referred to as superabsorbents; and the like. Other absorbent materials such as foams may also be used. Non-limiting examples of suitable foam absorbents are open celled foams such as cellulosic or polyether foams. Blends of two or more types of absorbent materials may be used to optimize the performance of fluid management articles used in varying conditions. For example, the absorbent means may comprise a blend of absorbent materials and thermoplastic fibers, to provide structural integrity to the formed structure or for heat sealability to the liquid impermeable layer. Useful thermoplastic fibers are polyolefins, such as polypropylene and polyethylene fibers. The thermoplastic fibers may be bi-component or multi-component fibers having a first component having a first melting temperature and two or more additional components having different melting temperatures to that of the first melting temperature. Bi-component fibers are typically configured sheath-core or side-by-side. Suitable bi-component fibers include polyester/polyethylene and polypropylene/polyethylene.

In a preferred example the absorbent means is a material comprising cellulosic fibers and superabsorbent powder. The material preferably contains from about 40 to about 95 weight percent cellulosic fibers and, more preferably from about 60 to about 80 weight percent cellulosic fibers. Such a material may contain from about 5 to about 60 weight percent superabsorbent powder (SAP), preferably from about 20 to about 55 weight percent SAP, even more preferably from about 30 to about 45 weight percent SAP, and most preferably about 40 weight percent SAP.

While particular embodiments of the present invention have been illustrated and described, it would be obvious to those skilled in the art that various other changes and modifications can be made without departing from the spirit and scope of the invention.

Test Methods

Caliper:

As indicated above, the flexible aqueous liquid absorbing structure preferably has a thickness or caliper of about 4 mm or less. The apparatus required to measure the thickness of the sanitary napkin is a footed dial (thickness) gauge, Model No. ADP1116, available from B. C. Ames Company, of Waltham, Mass., with foot 29.6 mm diameter with stand, 56.7 gm. deadweight accurate to 0.0254 mm. A digital type apparatus is preferred. If the aqueous liquid absorbing structure sample is individually folded and/or wrapped, the sample is unwrapped and carefully flattened by hand prior to measurement. The foot of the gauge is raised and the sample is placed on the anvil such that the foot of the gauge is approximately centered to the sample (or in the location of interest on the sample of interest). When lowering the foot, care is taken to avoid allowing the foot to “drop” or that undue force is not applied. The read out is allowed to stabilize for approximately 5 seconds. The thickness reading is then taken. For each sample being tested, five (5) readings are recorded and an average is calculated and reported as the average caliper in mm.

Modified Circular Bend Stiffness

Apparatus

The apparatus necessary for the CIRCULAR BEND PROCEDURE is a modified Circular Bend Stiffness Tester, having the following parts:

A smooth-polished steel plate platform which is 102.0.times.102.0.times.6.35 millimeters having an 18.75 millimeter diameter orifice. The lap edge of the orifice should be at a 45 degree angle to a depth of 4.75 millimeters.

A plunger having an overall length of 72.2 millimeters, a diameter of 6.25 millimeters, a ball nose having a radius of 2.97 millimeters and a needle-point extending 0.88 millimeter therefrom having a 0.33 millimeter base diameter and a point having a radius of less than 0.5 millimeter, the plunger being mounted concentric with the orifice and having equal clearance on all sides. Note that the needle-point is merely to prevent lateral movement of the test specimen during testing. Therefore, if the needle-point significantly adversely affects the test specimen (for example, punctures an inflatable structure), than the needle-point should not be used. The bottom of the plunger should be set well above the top of the orifice plate. From this position, the downward stroke of the ball nose is to the exact bottom of the plate orifice.

A force-measurement gauge and more specifically an Instron inverted compression load cell. The load cell has a load range of from about 0.0 to about 2000.0 grams.

An actuator, and more specifically the Instron Model No. 1122 having an inverted compression load cell. The Instron 1122 is made by the Instron Engineering Corporation, Canton, Mass.

Number and Preparation of Specimens

In order to perform the procedure for this test, as explained below, five representative sanitary napkins are necessary. From one of the five napkins to be tested, some number “Y” of 37.5.times.37.5 millimeter test specimens are cut. Specimens having portions in which a liquid permeable cover layer is joined directly to a barrier sheet or which are a laminate of a liquid permeable cover layer, two or less tissue sheets and a barrier means, should not be tested. The reason that these specimens are not tested is due to the realization that prior art napkins exist in which a liquid permeable cover layer is joined to a barrier means beyond the edges of an absorbent material in the periphery of the napkin, such portions of which are highly flexible. However, the present invention is more concerned with the overall flexibility of the sanitary napkin and not merely the peripheral portions thereof and, therefore, the flexibility of the present invention is more concerned with the flexibility of the significant absorbent portions of the sanitary napkin. If any of these significant absorbent portions of the sanitary napkin meet the parameters of this test, then the sanitary napkin satisfies the test. Therefore, a number of different specimens should be tested from each sanitary napkin. Certainly, the structurally most flexible portion of the sanitary napkin should be tested, excluding those portions excluded above. The test specimens should not be folded or bent by the test person, and the handling of specimens must be kept to a minimum and to the edges to avoid affecting flexural-resistance properties. From the four remaining sanitary napkins, an equal number “Y” of 37.5.times.37.5 millimeter specimens, identical to the specimens cut from the first napkin, are cut. Thus, the test person should have “Y” number of sets of five identical specimens.

Procedure

The procedure for the CIRCULAR BEND PROCEDURE is as follows. The specimens are conditioned by leaving them in a room which is 21.+−.1.degree. C. and 50.+−.2% relative humidity for a period of two hours. The test plate is leveled. The plunger speed is set at 50.0 centimeters per minute per full stroke length. A specimen is centered on the orifice platform below the plunger such that the body surface 26 of the specimen is facing the plunger and the garment surface 17 of the specimen is facing the platform. The indicator zero is checked and adjusted, if necessary. The plunger is actuated. Touching the specimen during the testing should be avoided. The maximum force reading to the nearest gram is recorded. The above steps are repeated until all five of the identical specimens have been tested.

Calculations

The peak bending stiffness for each specimen is the maximum force reading for that specimen. Remember that “Y” number of sets of five identical specimens were cut. Each set of five identical specimens is tested and the five values received for that set are averaged. Thus, the test person now has an average value for each of the “Y” sets tested. Remember, if any of the significantly absorbent portions of the sanitary napkin have the requisite flexural resistance, then the napkin satisfies the parameters of this test. Therefore, the flexural resistance for a particularly designed sanitary napkin is the greatest of these average peak bending stiffnesses.

Gurley Stiffness:

Gurley stiffness is one of many indices of softness. Gurley stiffness measures the bendability or flexibility of absorbent materials. The lower the Gurley stiffness value, the more flexible the material. The Gurley stiffness values are measured using a Gurley Stiffness Tester (Model No. 4171D), manufactured by Gurley Precision Instruments of Troy, N.Y. The instrument measures the externally applied moment required to produce a given deflection of a test strip of specific dimensions fixed at one end and having a concentrated load applied to the other end. The results are obtained in “Gurley Stiffness” values in units of milligrams. The procedure for determining Gurley Stiffness is as follows:

1. Locate Y areas of the product being tested that contain(s) the aqueous liquid absorbing material (ALAM) and mark these areas for identification (eg. Zone 1, zone 2, zone 3, etc.).

2. For each different area, die cut a rectangular sample 25.0 millimeters (mm) wide by 50.0 mm long.

Note: If the dimensions of the the Gurley tester is 12 mm wide by 24 mm long.)ALAM area are less 25.0 by 50.0 (mm), die cut the next largest sample size possible by reducing the rectangle dimensions by increments of 12 mm. (Note the smallest size permitted by

3. Remove any release paper that may exist on the sample(s) and eliminate any stickiness that may exist by applying a layer of talcum powder and smoothing out any wrinkles.

4. Calibrate the Gurley tester per manufacturer's instructions.

5. Position the arm on the Gurley apparatus to the right.

6. According to the manufacturer's instructions, insert one sample at a time into the clamp and secure sample so the longer edge of the sample is parallel to the clamp.

7. Make sure the pendulum is steady at zero and move the switch to the left so that the entire sample moves across the pendulum.

8. Re-steady the pendulum at zero and move the switch to the right so that the entire sample moves across the pendulum.

9. Record the digital stiffness reading.

10. Repeat steps 1-10 until n=5 for each of Y zones identified (n=5 for zone 1, n=5 for zone 2, etc.).

11. Average the readings from each zone separately and report the lowest average reading as the stiffness value for the sample being tested.

Absorbency:

The absorbency of the flexible aqueous liquid absorbing structure of the present invention and of products containing the structure is determined using the Tea Bag Test. The Tea Bag Test determines the freeswell absorbency (Tea Bag Capacity ) and results are expressed as grams of liquid absorbed per gram of dry aqueous liquid absorbing material (ALAM) being tested (g/g) The procedure for applying the Tea Bag Test to structures and articles of the present invention is as follows:

Determination of Dry Weight of ALAM:

1. Locate Y absorbent areas from the structure containing the aqueous liquid absorbing material (ALAM) or of the product being tested

2. Die cut a circular sample 60 mm in diameter or, if this size sample cannot be cut, cut a sample of equivalent area (2828 mm²) from the ALAM.

3. Physically strip off as many layers from structure or product without disturbing the layer containing ALAM and weigh sample.

4. If further isolation of the ALAM can be obtained through chemical stripping, separate ALAM and weigh the remaining material.

5. The dry ALAM weight is found by subtracting the weight found in step 4 from the weight found in step 3. If step 4 was not possible then the dry ALAM weight is found in step 3.

6. Repeat steps 1-5 until n=5 for each of the Y areas.

7. Calculate the dry weight of ALAM for each area as the average of 5 measurements.

8. The lowest average is reported as the dry weight of ALAM (E)

Determination of Liquid Absorbed by Structure or Products with ALAM:

1. Locate the Y most absorbent areas from the structure containing the aqueous liquid absorbing material (ALAM) or of the product being tested

2. Die cut a circular sample 60 mm in diameter or, if this size sample cannot be cut, cut a sample of equivalent area (2828 mm²) from the ALAM.

3. Die cut two 70×140-mm rectangular tea bag fabric pieces (tea bag fabric is Code 01234T9, produced by Ahistrom-Dexter Corporation, Windsor Locks, Conn.).

4. Encase the die cut sample to be tested in one of the tea bags by folding and heat sealing the tea bag on 3 sides approximately 3 mm from the edge.

5. Fold and heat seal the second tea bag in the same manner without sample; this will serve as a blank.

6. Record the weights of both tea bags.

7. Place both sealed tea bags into a container with 0.9% saline solution for 30 minutes (The tea bags should be submerged).

8. After 30 minutes, take the sample tea bag and the blank tea bag out of the solution and hang vertically with clips for 10 minutes (Be sure to clip the sample by the tea bag fabric only).

9. After 10 minutes, weigh and record the wet weights of both tea bags.

10. Calculate the gram/gram capacity of the sample as follows:

Absorbency or Tea Bag Capacity (g/g)=(A−B−(C−D))/E

Where A=Wet weight of sample

B=Wet weight of blank

C=Dry weight of sample with tea bag

D=Dry weight of blank tea bag

E=Dry weight of ALAM

11. Repeat steps 1-10 until n=5 for each of Y areas.

12. Calculate the absorbency for each area as the average of 5 measurements.

13. The highest average is reported as the tea bag capacity or Absorbency of the sample being tested.

Thickness Under Load

Overview:

This test procedure determines material thickness as a function of applied load. Data generated using this procedure can he used to calculate compressibility and compression resistance as functions of applied load.

Scope:

This procedure applies to materials from 0.2 mm to 5 mm thick. Samples tested by this procedure should be flat and not have significant curvature. Top and bottom surfaces of the material should be essentially parallel.

Sampling:

Samples should be cut such that they are larger than the 25.4 mm diameter platen used to compress the sample. A rectangular cutting die 3.81 cm by 6.99 cm is suitable. The area to be tested must be able to lie flat. Samples may be cut from individual materials or finished products but the cut area must be free from wrinkles or curvature and the sampling procedure should not distort the dimensions of the material. For example, when sampling finished products, the sample material must be cleanly separable from other materials. At least three different samples should be measured with the average result reported.

Apparatus:

Use a constant rate of extension (CRE) tester. A suitable CRE tester is the Instron Model 5564, available from the Instron Corporation, Canton, Mass. The CRE tester must have accurate control and measurement of crosshead position to 0.01 mm. The CRE tester must be able to measure load accuracy to 0.5 gram force (5 mN). The CRE tester should have less than 0.02 mm compliance at IN applied load. The moving crosshead should be fitted with a 25.4 mm diameter (5.07 cm² area) circular, flat platen disk, rigidly attached to the load cell. A circular, flat platen at least 100 mm in diameter should be rigidly fixed to the lower stationary clamp. The faces should be parallel with each other with a tolerance of 0.02 mm or less.

Procedure:

1. The grammage of the sample should be determined by weighing a precisely known area of 100 cm² or larger on an analytical balance to the nearest 0.0002 g.

2. With the parallel platens a known distance apart, the flat sample is placed on the lower platen. For a 1.00 mm thick sample, 3.00 mm is the recommended starting separation. The CRE load cell should be zeroed at the start of each test while the platens are not contacting the sample.

3. The CRE tester lowers the upper platen toward the sample at a rate of 2.54 mm per minute, recording at least 100 data points per mm of travel, until the sample is compressed and a load of 20 kilograms (kg) force is reached.

The thickness of the material at any given load is determined by the position of the upper platen. Thickness should be measured at a load of 10 kg grams force and 20 kg force.

Calculations:

Compressibility is determined by calculating the secant slope of the stress/strain compression curve between 10 kg and 20 kg and dividing this slope by the area compressed. Units for compressibility are kilograms (kg) per cm². This slope is calculated by dividing the change in load by the change in thickness. For example, if a material changed in thickness by 0.014 mm as the load changed from 10 kg to 20 kg:

$$\frac{20 kg - 10 kg}{0.014 mm} = 714 kg \text{/} mm Compressibility$$

Similarly, Compression Resistance is calculated by dividing the compressibility by the area of the sample tested, in cm².

$$Compression Resistance = \frac{Compressibility}{Area}$$

Test and Total Capacity

The test and total capacities of a sanitary napkin are determined as follows. Any panty adhesive release paper is removed from the napkin to be tested. To determine test capacity, a 4.75.times. 14.0 centimeters portion, or any other configuration having 66.5 square centimeters, of the sanitary napkin is cut from the portion of the sanitary napkin which would be centered under the vaginal orifice when the sanitary napkin is worn. Total capacity is determined using the entire napkin minus any release paper. The article is weighed to the nearest 0.1 gram. The article is then submerged in a beaker of sterile saline (obtainable from the Baxter Travenol Company of Deerfield, Ill.), such that the article is totally submerged and is not bent or otherwise twisted or folded. The article is submerged for 10 minutes. The article is removed from the saline and suspended for two minutes in a vertical position to allow the saline to drain out to the article. The article is then placed body faceable surface down onto an absorbent blotter, such as the filter paper #631 available from the Filtration Science Corp., Eaton-Dikeman Division of Mount Holly Springs, Pa. A uniform 17.6 grams per square centimeter load is placed over the article to squeeze excess fluid out. The absorbent blotter is replaced every 30 seconds until the amount of fluid transferred to the absorbent blotter is less than 0.5 grams in a 30 second period. Next, the article is weighed to the nearest 0.1 gram and the dry weight of the article is subtracted. The difference in grams is the test or total capacity of the article, whichever the case may be.

While particular embodiments of the present invention have been illustrated and described, it would be obvious to those skilled in the art that various other changes and modifications can be made without departing from the spirit and scope of the invention.

We claim:

1. A sanitary napkin for feminine hygiene adapted for placement in a crotch portion of an undergarment, having a body surface and a garment surface comprising: a liquid permeable cover layer, a liquid impermeable barrier means and an absorbent means between the cover layer and the barrier means, wherein the absorbent means has a compression resistance greater than about 110 kg/cm³; and said sanitary napkin having a flexural resistance of less than about 400.0 grams, a test capacity of at least about 5.0 grams, and a total capacity of at least about 20.0 grams.

2. The sanitary napkin as claimed in claim 1, wherein said sanitary napkin has a flexural resistance of less than about 250.0 grams.

3. The sanitary napkin as claimed in claim 1, wherein said sanitary napkin has a caliper 12 of about 4 millimeters or less.

4. The sanitary napkin as claimed in claim 1, wherein said sanitary napkin has a caliper 12 of about 3 millimeters or less.

5. The sanitary napkin as claimed in claim 1, wherein said sanitary napkin has a caliper 12 of about 2.2 millimeters or less.

6. The sanitary napkin as claimed in claim 3, wherein said absorbent means has a compression resistance greater than 250 kg/cm³.

7. The sanitary napkin as claimed in claim 3, wherein said sanitary napkin has a flexural resistance of less than about 175.0 grams.

8. The sanitary napkin as claimed in claim 3, wherein said sanitary napkin has a test capacity of at least about 15.0 grams.

9. The sanitary napkin as claimed in claim 3, wherein said sanitary napkin has a flexural resistance of less than about 130.0 grams.

10. The sanitary napkin as claimed in claim 3, wherein at least a portion of the absorbent means further comprises a hydrogel-forming polymer gelling agent.

11. The sanitary napkin as claimed in claim 10, wherein said absorbent means comprises an absorbent material, and wherein said absorbent material contains from about 5% to about 85% by weight of a particulate hydrogel-forming polymer gelling agent.

12. The sanitary napkin as claimed in claim 3, wherein said absorbent means comprises a transfer layer overlaying an absorbent material.

13. The sanitary napkin as claimed in claim 3, wherein said absorbent means further comprises a hydrogel-forming polymer gelling agent and a transfer layer, and the cover layer comprises an apertured formed film.

14. The sanitary napkin as claimed in claim 3, wherein said barrier means comprises a barrier sheet and said absorbent means comprises a liquid permeable polymeric topsheet overlaying an absorbent material, said topsheet being associated with said barrier sheet.

15. A sanitary napkin for feminine hygiene adapted for placement in a crotch portion of an undergarment and having a longitudinal axis, said sanitary napkin comprising:
a liquid permeable body faceable layer;
a liquid impermeable garment faceable layer;
an absorbent means positioned between said body faceable layer and said garment faceable layer, said absorbent means having a longitudinally-extending central absorbent zone aligned along the longitudinal axis of said sanitary napkin, said central absorbent zone having two opposing longitudinal edges and two opposing transverse edges; and
a peripheral zone aligned adjacent to one of said longitudinal edges of said absorbent zone, said peripheral zone comprising said body faceable layer, said garment faceable layer and an absorbent material therebetween; said peripheral zone being characterized by an absorbency greater than about 5 g/g, a caliper less than about 3 mm, and a compression resistance greater than about 10 kg/cm³;
the central absorbent zone and the peripheral zone having a respective Gurley stiffness wherein the Gurley stiffness of the peripheral zone is less than the Gurley stiffness of the central absorbent zone.
